(19) United States
(12) Patent Application Publication
(10) Pub. No.: US 2010/0154889 A1
(43) Pub. Date: Jun. 24, 2010

(54) ELECTROLYTE COMPOSITION AND DYE-SENSITIZED SOLAR CELL USING THE SAME

(75) Inventors: Yung-Liang Tung, Hualien County (TW); Jia-Yin Wu, Taichung County (TW); Jen-An Chen, Miaoli County (TW); Wen-Yueh Ho, Tainan (TW); Yu Kai Chou, Taipei (TW)

Correspondence Address: PAI PATENT & TRADEMARK LAW FIRM, 1001 FOURTH AVENUE, SUITE 3200, SEATTLE, WA, 98154, US

(73) Assignee: INDUSTRIAL TECHNOLOGY RESEARCH INSTITUTE, Hsinchu County (TW)

(21) Appl. No.: 12/463,395

(22) Filed: May 9, 2009

(30) Foreign Application Priority Data
Dec. 19, 2008 (TW) 97149632

(51) Int. Cl. H01L 31/00; H01G 9/022; C07D 403/12

(52) U.S. Cl. 136/261; 548/313.7; 252/62.2

(57) ABSTRACT

The invention provides an electrolyte composition and dye-sensitized solar cell using the same. The electrolyte composition includes a diionic liquid of Formula: Z⁻ (X—Y—X)Z⁻, wherein X includes ammonium, imidazolium, pyridinium or phosphonium, Y is (CH₂)n, n is an integer of 1-16, Z is I, and Z′ is I, PF₆, BF₄, N(SO₂CF₃), NCS or N(CN)₂.

### CROSS REFERENCE TO RELATED APPLICATIONS

This Application claims priority of Taiwan Patent Application No. 097149632, filed on Dec. 19, 2008, the entirety of which is incorporated by reference herein.

### BACKGROUND OF THE INVENTION

1. Field of the Invention

The present invention relates to an electrolyte composition, and in particular relates to an electrolyte composition used in dye-sensitized solar cells.

2. Description of the Related Art

Development in the solar cell industry is driven by global environmental concerns and rising raw material prices. Among the various solar cells developed, dye-sensitized solar cell (DSSC) is advantageous as it can be fabricated with relatively lower costs due to its simpler fabrication process and ability for large area fabrication.

Conventionally, DSSCs used an I⁻/I₃⁻ electrolyte solution having poisonous and volatile organic solvent, thus, causing great concern for fluid leakage. Thus, electrolyte compositions containing ionic liquid have been disclosed, in hopes to replace the I⁻/I₃⁻ electrolyte solution. Advantageous of electrolyte compositions containing ionic liquid include it being non-poisonous and less volatile, having a low melting point, non-flammatory, and having high thermal stability.

US patent publication NO. 2006/0174932 provides an electrolyte composition containing ionic liquid and conductive particles, wherein the ionic liquid is 1-ethyl-3-methylimidazolium iodide.

US patent publication NO. 2008/0060698 provides an electrolyte composition and photoelectric conversion element utilizing the same, wherein the electrolyte composition comprises an ionic liquid having dicyanoamide anions.

Nevertheless, current disclosed electrolyte compositions containing ionic liquid still contain drawbacks such as fluid leakage solvent volatilization. Thus, there is a need to develop a novel ionic liquid to improve the disadvantages of conventional electrolyte compositions.

### BRIEF SUMMARY OF THE INVENTION

The invention provides an electrolyte composition, comprising a diionic liquid having a Formula: Z⁻(X—Y—X)Z⁻, wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

The invention also provides a dye-sensitized solar cell, comprising: a working electrode, wherein the working electrode comprises an electrode substrate and an oxide semiconductor porous film formed on the substrate; a counter electrode disposed oppositely from the working electrode; an electrolyte composition provided between the working electrode and the counter electrode, wherein the electrolyte composition comprises a diionic liquid having a Formula: Z⁻(X—Y—X)Z⁻, wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

A detailed description is given in the following embodiments with reference to the accompanying drawings.

### BRIEF DESCRIPTION OF THE DRAWING

For a more complete understanding of the present invention, and the advantages thereof, reference is now made to the following descriptions taken in conjunction with the accompanying drawings, in which:

FIG. 1 shows a cross-sectional schematic representation of a dye-sensitized solar cell of the invention; and

FIG. 2 shows a long term stability test of a dye-sensitized solar cell of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides an electrolyte composition, comprising a diionic liquid having a Formula:

Z⁻(X—Y—X)Z′⁻

, wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

In one embodiment, the diionic liquid has a Formula as shown below:

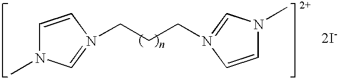

, wherein n is an integer of 1-16, preferably an integer of 3-8.

The electrolyte composition of the invention further comprises other additives, such as solvents, stabilizers or soluble electrolytes. The solvent comprises ethanol, acetonitrile, methoxy acetonitrile, propionitrile, 3-methoxypropionitrile (MPN), ethyl carbonate, propyl carbonate, ethylene carbonate or 2-ethyl-4-methyl imidazole.

The above mentioned stabilizer comprises lithium iodide (LiI), N-butyl benzimidazole (NBB), 1-methylbenzimida (NMBI) or 4-tert-butyl pyridine (TBP). The purpose of the stabilizer is to improve the stability the iodide of the diionic liquid. The concentration of the stabilizer is about 0.05 M to 1.0 M, preferably about 0.1 M to 0.3 M.

The above mentioned soluble electrolyte comprises 1-propyl-3-methyl imidazolium iodide (PMII) [symbol] 1-ethyl-3-methylimidazolium iodide (EMII), 1,3-dimethyl imidazolium iodide (DMII) or 1-allyl-3-methylimidazolium iodide (AMII). The purpose of the soluble electrolyte is to help the oxidation-reduction reaction. The sum of the concentration of the soluble electrolyte and diionic liquid is about 0.1 M to 1.0 M.

The main feature of the diionic liquid of the invention is that each side of the diionic liquid has ions, thus the sides of the diionic liquid may be a symmetric or asymmetric structure. When both sides are asymmetric structures, the oxidation-reduction reaction may be conducted on one side with the other side used as the solvent instead of the original organic solvent. The electrolyte composition of the invention may be applied in a photoelectric conversion element, such as dye-sensitized solar cell (DSSC).

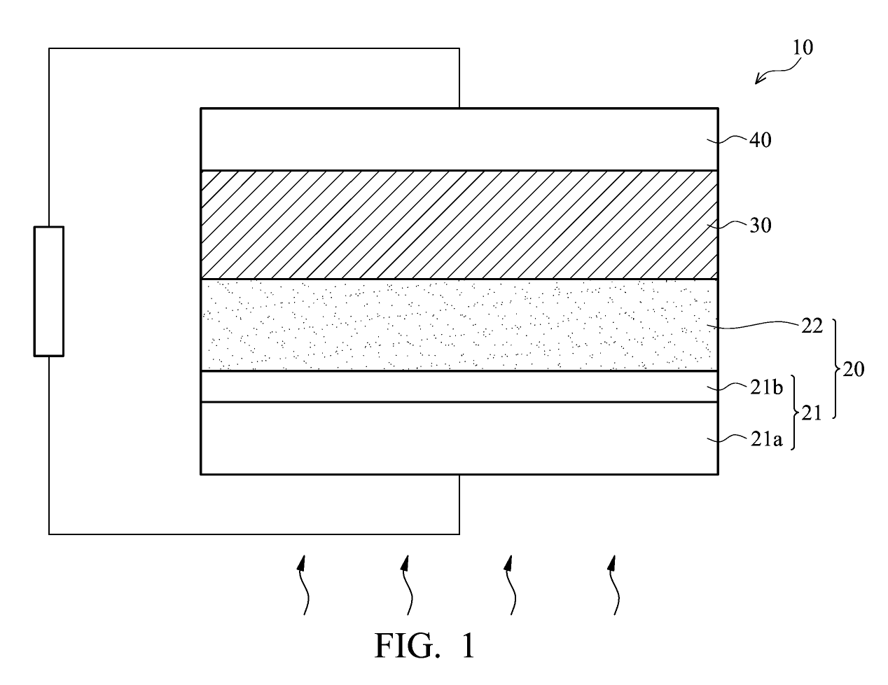

The invention also provides a dye-sensitized solar cell as shown in FIG. 1. The dye-sensitized solar cell 10 comprises a working electrode 20, a counter electrode 40 disposed oppositely to the working electrode 20, and an electrolyte composition 30 provided between the working electrode 20 and the counter electrode 40, wherein the electrolyte composition 30 comprises a diionic liquid having a Formula:

Z⁻(X—Y—X)Z′⁻

, wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

In one embodiment, the diionic liquid has a Formula as shown below:

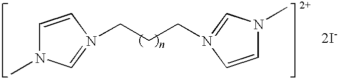

, wherein n is an integer of 1-16, preferably an integer of 3-8.

The electrolyte composition of the invention further comprises other additives, such as solvents, stabilizers or soluble electrolytes, which are the same as those in the first embodiment, and thus further description is omitted for brevity.

The working electrode 20 comprises an electrode substrate 21 and an oxide semiconductor porous film 22 formed on the electrode substrate 21, wherein the electrode substrate 21 comprises a transparent substrate 21a and a conductive layer 21b formed on the transparent substrate 21a. The transparent substrate 21a comprises glass, a transparent plastic sheet or polished ceramic plate. The conductive layer 21b comprises tin-doped indium oxide (ITO), tin oxide (SnO₂), fluorine-doped tin oxide (FTO) or combinations thereof. The oxide semiconductor porous film 22 comprises titanium oxide (TiO₂), tin oxide (SnO₂), tungsten oxide (WO₃), zinc oxide (ZnO), niobium oxide (Nb₂O₅) or combinations thereof. In one embodiment, the oxide semiconductor porous film 22 is preferably titanium oxide (TiO₂).

The purpose of the oxide semiconductor porous film 22 is to absorb dye. The dye comprises Ru complexes such as N3 dye (Ru(NCS)₂) or N719 dye (RuL₂(NCS)₂: 2TBA), porphyrin, phthalocyanine or coumarin.

The above mentioned counter electrode 40 comprises a non-conductive substrate and a conductive film formed on the non-conductive substrate. For example, platinum film, carbon film or the like is formed on the ITO, FTO or like substrates.

The diionic liquid of the invention is used as an electrolyte composition in a dye-sensitized solar cell (DSSC). The photoelectric conversion efficiency of a DSSC is about greater than 5%. The photoelectric conversion efficiency of the DSSC of the invention is close to that of the conventional DSSCs in which 1-methyl-3-propylimidazolium iodide (PMII) was used in the electrolyte composition. In addition, the concentration of the electrolyte composition of the invention is about 0.1 M to 1.0 M, which is lower than that in the conventional DSSC (about 0.4 M to 1.5 M). In one embodiment, the concentration of the electrolyte composition of the invention is only 0.22 M, thus the photoelectric conversion efficiency of the DSSC of the invention may be equivalent to that of 0.8 M PMII.

In addition, the DSSC of the invention was measured by a long term stability test. The experiment was performed at 60° C. and under sunlight of 1000 W/m². The initial efficiency of the DSSC was set as 100%, and after a period of time the DSSC was measured to record efficiency decay. The results showed that the efficiency decay of the invention was between 60% and 70% after 1200 hours. However, the efficiency decay of the conventional 1-methyl-3-propylimidazolium iodide (PMII) was about 50%. In comparison, the diionic liquid of the invention improved the stability of DSSC, especially for long term stability.

The diionic liquid of the invention has several advantages:

(1) When the diionic liquid is an asymmetric structure, the oxidation-reduction reactions may be conducted on one side with the other side used as a solvent instead of the original organic solvent.

(2) The photoelectric conversion efficiency of the DSSC of the invention is close to that of the conventional DSSC in which 1-methyl-3-propylimidazolium iodide (PMII) was used in the electrolyte composition.

(3) The stability of the DSSC of the invention is higher than the conventional DSSC.

### PREPARATIVE EXAMPLE

There are three diionic liquids shown in Table 1:

TABLE 1

| | an abbreviation | |
|---|---|---|
| compound | of diionic liquid | n |
| 2a | ImC₃Im2I | 1 |
| 2b | ImC₅Im2I | 3 |
| 2c | ImC₁₀Im2I | 8 |

The synthetic steps of compound 2a, 2b and 2c are shown below:

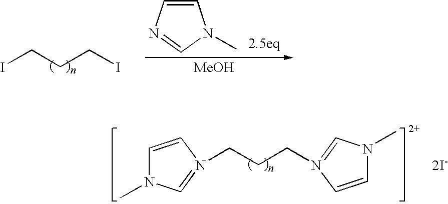

### Preparative Example 1

### Synthesis of Compound 2a

2.96 g (10.0 mmol) of 1,3-diiodopropane was slowly added into an ice bath of 2.05 g (25.0 mmol) of 1-methylimidazole and 10 ml of methanol. The mixture was heated at 70° C. for 24 hours and then cooled to room temperature. Next, the mixture was added into 50 ml of ethyl acetate to produce a large amount of a white solid. The white solid was filtered and then cleaned by ethyl acetate. The filtrate was dried to obtain compound 2a. (4.5g, 98%)

NMR data of the compound 2a was as follows.

1H NMR(200 Hz, D₂O): 7.49 (s, 2H), 7.43(s, 2H), 4.29(t, 4H, J=6.8 Hz), 3.86(s, 6H), 2.53-2.46(m, 2H).

### Preparative Example 2

### Synthesis of Compound 2b

16.20 g (50.0 mmol) of 1,5-diiodopentane was slowly added into an ice bath of 10.25 g (125.0 mmol) of 1-methylimidazole and 50 ml of methanol. The mixture was heated at 70° C. for 48 hours and then cooled to room temperature. Next, the mixture was added into 50 ml of ethyl acetate to produce a large amount of a white solid. The white solid was filtered and then cleaned by ethyl acetate. The filtrate was dried to obtain compound 2b. (22g, 90%)

NMR data of the compound 2b was as follows

1H NMR(200 Hz, D₂O): 8.68(s, 2H), 7.43(s, 2H), 7.39(s, 2H), 4.16(t, 4H, J=7.2 Hz), 3.85(s, 6H), 1.92-1.84(m, 4H), 1.33-1.28(m, 2H).

### Preparative Example 3

### Synthesis of Compound 2c

15.6 g (39.6 mmol) of 1,10-diiododecane was slowly added into an ice bath of 8.12 g (99.0 mmol) of 1-methylimidazole and 40 ml of methanol. The mixture was heated at 70° C. for 48 hours and then cooled to room temperature. Next, the mixture was added into 200 ml of ethyl acetate to produce a large amount of a white solid. The white solid was filtered and then cleaned by ethyl acetate. The filtrate was dried to obtain compound 2c. (20g, 91%)

NMR data of the compound 2c was as follows.

1H NMR(200 Hz, D₂O): 8.68(s, 2H), 7.44(s, 2H), 7.39(s, 2H), 4.15(t, 4H, J=6.9 Hz), 3.85(s, 6H), 1.94-1.85(m, 4H), 1.38-1.02(m, 12H).

### EXAMPLE

### Example 1

### Comparison of Photoelectric Conversion Efficiency

Referring to FIG. 1, a glass with ITO was used as an electrode substrate 21, TiO₂ with N719 dye was used as an oxide semiconductor porous film 22, and platinum with ITO was used as a counter electrode 40. The electrolyte composition 30 is shown in Table 2, and the 3-methoxypropionitrile (MPN) was used as solvent. Additionally, each of the electrolyte compositions further comprised 0.05 M I₂ and 0.5 M 1-methylbenzimida.

TABLE 2

| | short-circuit | | | photoelectric |
|---|---|---|---|---|
| electrolyte | current | open-circuit | | conversion |
| composition | (mA/cm²) | voltage (V) | fill factor | efficiency (%) |
| 0.8 M PMII*¹ | 11.639 | 0.738 | 0.606 | 5.207 |
| (comparative | | | | |
| example) | | | | |
| 0.22 M 2c | 13.305 | 0.699 | 0.549 | 5.121 |
| 0.22 M 2c + | 14.393 | 0.686 | 0.521 | 5.148 |
| 0.58 M PMII*¹ | | | | |
| 0.1 M 2b + | 12.608 | 0.693 | 0.603 | 5.270 |
| 0.7 M PMII*¹ | | | | |

*¹PMII(1-methyl-3-propylimidazolium iodide, PMII) is a conventional ionic liquid.

As shown in Table 2, the photoelectric conversion efficiency of the invention was close to that of the conventional DSSC in which 1-methyl-3-propylimidazolium iodide (PMII) was used in the electrolyte composition. Furthermore, the concentration of the electrolyte composition of compound 2c of the invention was only 0.22 M, thus the photoelectric conversion efficiency of the DSSC of the invention may be equivalent to 0.8 M PMII. Thus, the amount of electrolyte composition can be reduced. And, when the diionic liquid of the invention was used with the conventional PMII in an electrolyte composition, the DSSC also exhibited good photoelectric conversion efficiency.

### Example 2

### Long Term Stability Test

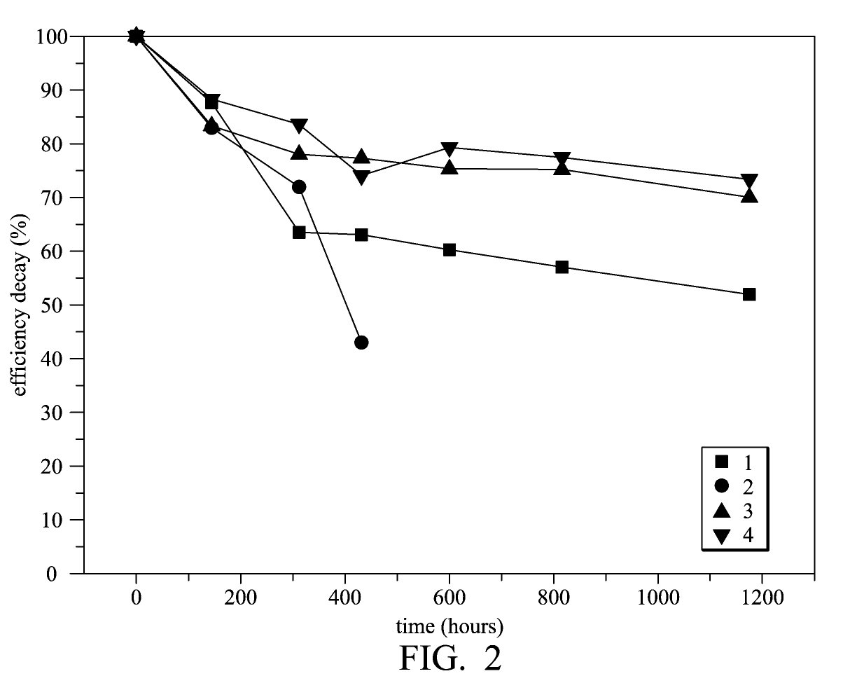

The electrolyte compositions in Table 3 were measured by a long term stability test. The experiment was performed at 60° C. and under sunlight of 1000 W/m². The efficiency decay of DSSC is shown in Table 3 and the time VS. efficiency decay is shown in FIG. 2.

TABLE 3

| | | efficiency |
|---|---|---|
| | | decay |
| sample | electrolyte composition | (%) |
| 1 | 0.6 M PMII/0.05 M I₂/0.1 M LiI/0.5 M | 51.9 |
| | TBP*²/MPN*³ | |
| 2 | the same as sample 1 | 43.0 |
| 3 | 0.436 M PMII/0.05 M I₂/0.1 M LiI/0.125 M | 70.0 |
| | TBP*²/saturated 2b/3.4 g EMINTF2*⁴/MPN*³ | |
| 4 | the same as sample 3 | 73.4 |

*²TBP: 4-tert-butyl pyridine
*³MPN: 3-methoxypropionitrile
*⁴EMINTF2: 1-ethyl-3-methylimidazolium bis(trifluoromethylsulfonyl)imide

As shown in Table 3 and FIG. 2, the efficiency decay of sample 1 after 1200 hours was about 50%, and efficiency decay of sample 1 after 400 hours was about 40%. However, the efficiency decay of sample 3 and sample 4 (the electrolyte composition of the invention) was only about 70%. Therefore, the diionic liquid of the invention improved the stability of DSSC, especially for long term stability.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An electrolyte composition, comprising a diionic liquid having a Formula:
Z⁻(X—Y—X)Z′⁻
,wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

2. The electrolyte composition as claimed in claim 1, wherein the diionic liquid has a Formula: 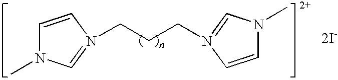
, wherein n is an integer of 1-16.

3. The electrolyte composition as claimed in claim 1, further comprising: a solvent, a stabilizer and a soluble electrolyte.

4. The electrolyte composition as claimed in claim 3, wherein the solvent comprises ethanol, acetonitrile, methoxy acetonitrile, propionitrile, 3-methoxypropionitrile (MPN), ethyl carbonate, propyl carbonate, ethylene carbonate or 2-ethyl-4-methyl imidazole.

5. The electrolyte composition as claimed in claim 3, wherein the stabilizer comprises lithium iodide (LiI), N-butyl benzimidazole (NBB), 1-methylbenzimida (NMBI) or 4-tert-butyl pyridine (TBP).

6. The electrolyte composition as claimed in claim 3, wherein the soluble electrolyte comprises 1-propyl-3-methyl imidazolium iodide (PMII), 1-ethyl-3-methyl imidazolium iodide (EMII), 1,3-dimethyl imidazolium iodide (DMII) or 1-allyl-3-methyl imidazolium iodide (AMII).

7. The electrolyte composition as claimed in claim 1, wherein the electrolyte composition is used in photoelectronic conversion devices.

8. The electrolyte composition as claimed in claim 7, wherein the photoelectronic conversion devices comprises dye-sensitized solar cells.

9. A dye-sensitized solar cell, comprising:
a working electrode, wherein the working electrode comprises an electrode substrate and an oxide semiconductor porous film formed on the electrode substrate;
a counter electrode disposed oppositely from the working electrode;
an electrolyte composition provided between the working electrode and the counter electrode, wherein the electrolyte composition comprises a diionic liquid having a Formula:
Z⁻(X—Y—X)Z′⁻
, wherein X is ammonium, imidazolium, pyridinium or phosphonium, Y is (CH₂)ₙ, n is an integer of 1-16, Z is I, and Z′ is I, PF₆, BF₄, N(SO₂CF₃), NCS or N(CN)₂.

10. The dye-sensitized solar cell as claimed in claim 9, wherein the diionic liquid has a Formula: 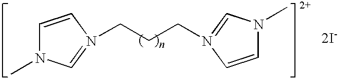
, wherein n is an integer of 1-16.

11. The dye-sensitized solar cell as claimed in claim 9, further comprising: a solvent, a stabilizer and a soluble electrolyte.

12. The dye-sensitized solar cell as claimed in claim 11, wherein the solvent comprises ethanol, acetonitrile, methoxy acetonitrile, propionitrile, 3-methoxypropionitrile (MPN), ethyl carbonate, propyl carbonate, ethylene carbonate or 2-ethyl-4-methyl imidazole.

13. The dye-sensitized solar cell as claimed in claim 11, wherein the stabilizer comprises lithium iodide (LiI), N-butyl benzimidazole (NBB), 1-methylbenzimida (NMBI) or 4-tert-butyl pyridine (TBP).

14. The dye-sensitized solar cell as claimed in claim 11, wherein the soluble electrolyte comprises 1-propyl-3-methyl imidazolium iodide (PMII), 1-ethyl-3-methyl imidazolium iodide (EMII), 1,3-dimethyl imidazolium iodide (DMII) or 1-allyl-3-methyl imidazolium iodide (AMII).

15. The dye-sensitized solar cell as claimed in claim 9, wherein the electrode substrate comprises a transparent substrate and a conductive layer formed on the transparent substrate.

16. The dye-sensitized solar cell as claimed in claim 15, wherein the transparent substrate comprises glass, a transparent plastic sheet or polished ceramic plate.

17. The dye-sensitized solar cell as claimed in claim 15, wherein the conductive layer comprises tin-doped indium oxide (ITO), tin oxide (SnO₂), fluorine-doped tin oxide (FTO) or combinations thereof.

18. The dye-sensitized solar cell as claimed in claim 9, wherein the oxide semiconductor porous film comprises titanium oxide (TiO₂), tin oxide (SnO₂), tungsten oxide (WO₃), zinc oxide (ZnO), niobium oxide (Nb₂O₅) or combinations thereof.

19. The dye-sensitized solar cell as claimed in claim 9, wherein the dye-sensitized solar cell has a photoelectric conversion efficiency greater than 5%.

20. The dye-sensitized solar cell as claimed in claim 19, wherein the diionic liquid has a concentration between 0.1 M to 1.0 M.
